(19) United States
(12) Patent Application Publication
(10) Pub. No.: US 2008/0234724 A1
(43) Pub. Date: Sep. 25, 2008

(54) Forceps for double eyelid operation

(76) Inventor: II-Bong Chung, Seoul (KR)

Correspondence Address: Thomas M. Galgano, Esq.;GALGANO & ASSOCIATES, PLLC, Suite 204, 20 W. Park Avenue, Long Beach, NY, 11561, US

(21) Appl. No.: 12/069,702

(22) Filed: Feb. 12, 2008

(30) Foreign Application Priority Data
Mar. 23, 2007 (KR) 20-2007-0004790
Jul. 9, 2007 (KR) 20-2007-0011236

(51) Int. Cl. A61B 17/28

(52) U.S. Cl. 606/207

(57) ABSTRACT

A forceps for a double eyelid. The forceps includes an upper support plate and a lower support plate. The upper support plate is attached to the front side of the handle and has one or more needles arranged in a front region of the inner surface of the upper support plate. The lower support plate corresponds to the upper support plate and is attached to the front side of the handle and having accommodation recesses corresponding to the needles and being formed in a front region of the inner surface of the lower support plate.

### CROSS-REFERENCE TO RELATED APPLICATION

This application claims priority to and the benefit of Korean Patent Application No. 20-2007-0004790 and 20-2007-0011236, filed on Mar. 23, 2007 and Jul. 09, 2007, the entire content of which is incorporated herein by reference.

### BACKGROUND OF THE INVENTION

1. Field of the Invention

The present invention relates to a tool for a double eyelid operation by a commonly used buried suture method, and more particularly, to a forceps for a double eyelid operation capable of accurately and simply performing the double eyelid operation by puncturing eyelids at several points on a desired double eyelid design line and simultaneously marking even conjunctiva portions corresponding to the points, without using a scalpel.

2. Description of the Related Art

In general, double eyelid operations are operations for connecting, by a thread, the skin (dermis) of an eyelid with the levator aponeurosis or the tarsal plate for use in opening an eye.

Double eyelid operations are classified into incisional methods performing an operation by incising the eyelid skin and buried suture methods performing an operation by using only a needle and a thread, without incising the eyelid skin. The incisional methods remove unnecessary muscles and fatty tissues after incising the eyelid skin, and the buried suture methods bind the dermis and the levator aponeurosis or tarsal plate together by using a suture needle and a thead, without completely incising the eyelid skin and bury the knots of the suture inside the skin.

Recently, the buried suture methods have been preferred since they do not incise the skin and leave few scars. However, in the conventional buried suture methods, it is necessary to individually incise several points of the skin where the suture is to be buried in a double eyelid line and it is difficult to accurately determine locations corresponding to the incised points of the skin even at the conjunctiva surface. Therefore, the operation time becomes longer and the operations themselves are not easy to unskilled surgeons.

### SUMMARY OF THE INVENTION

Therefore, the present invention has been made in view of the above problems, and it is an object of the present invention to provide a forceps for a double eyelid operation capable of conveniently and easily performing a buried suture method by puncturing the skin with needles such as sharp and small awls and marking at accurate locations on the tarsal plate that correspond to the penetrated locations of the skin, thereby using the accurate locations of the tarsal plate corresponding to the penetrated locations of the skin.

In order to achieve the above-mentioned object, the present invention provides a forceps for a double eyelid operation that can be resiliently regulated by handles easily holdable by a hand. The forceps includes an upper support plate and a lower support plate. The upper support plate is attached to the front side of the handle and has one or more needles arranged in a front region of the inner surface of the upper support plate. The lower support plate corresponds to the upper support plate and is attached to the front side of the handle and has accommodation recesses corresponding to the needles and being formed in a front region of the inner surface thereof.

A curved support member corresponding to a curved upper eyelid margin may be erected on and attached to a border between the accommodation recesses of the lower support plate and the handle such that the width between the needles arranged on the upper support plate and the support member can be kept constant when the lower support plate enters an eyelid and the needles nip the eyelid.

The needles of the upper support plate may be vertically formed from top to bottom or obliquely formed.

The accommodation recesses of the lower support plate may be vertically formed or obliquely formed and have blind-hole shapes or through-hole shapes.

The forceps according to the present invention can easily and conveniently perform a double eyelid operation by a buried suture method by inserting the lower support plate into the eyelid with the handle held by a hand and nipping incisional sites (holes) in a double eyelid line drawn on the eyelid skin with the needles of the upper support plate to form small holes on the skin surface and leave punctured marks on the tarsal plate.

According to the present invention, during a double eyelid operation by a buried suture method, since accurate locations of the tarsal plate corresponding to the incisional sites (small holes) can be accurately formed by puncturing the skin with needles formed in a forceps to form the small incisional sites in the eyelid skin, without using a scalpel, and by forming punctured marks at the accurate locations of the tarsal plate, the operation becomes simple and easy, thereby shortening the operation time. Therefore, the present invention provides convenience and facilitation for an operation to a surgeon and saves the time and the costs for the operation, resulting in economic and practical benefits and high efficiencies.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a perspective view illustrating a forceps for a double eyelid operation according to an embodiment of the present invention;

FIG. 2 is a longitudinal cross-sectional view illustrating main portions of the forceps of FIG. 1;

FIG. 3 is a longitudinal cross-sectional view of the forceps of FIG. 1, wherein upper and lower support plates of the forceps are engaged with each other;

FIG. 4 is a longitudinal cross-sectional view illustrating a forceps according to another embodiment of the present invention;

FIGS. 5A and 5B are longitudinal cross-sectional views illustrating forceps according to other embodiments of the present invention; and

FIG. 6 is a perspective view illustrating a forceps according to another embodiment of the present invention.

### DETAILED DESCRIPTION OF THE PREFERRED EMBODIMENTS

Hereinafter, preferred embodiments of the present invention are described in detail with reference to the accompanying drawings.

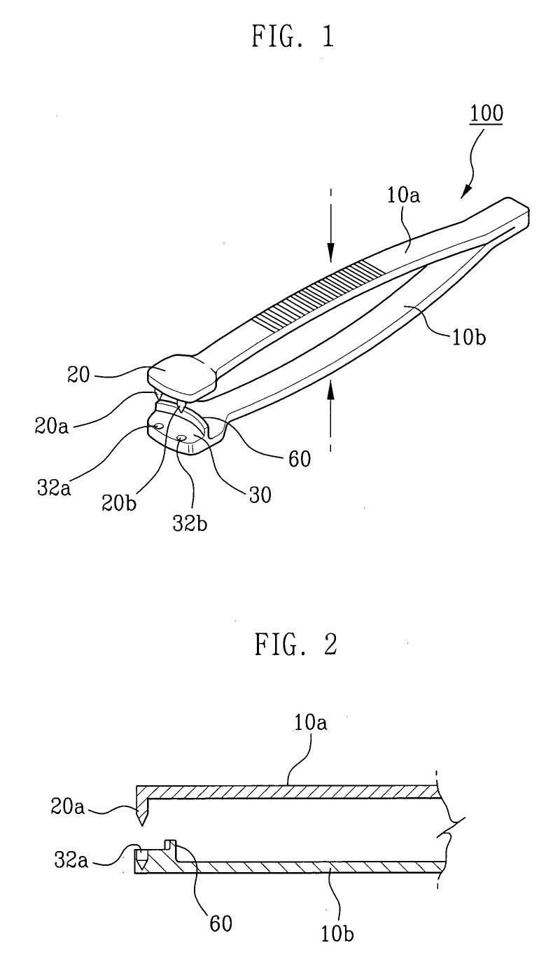

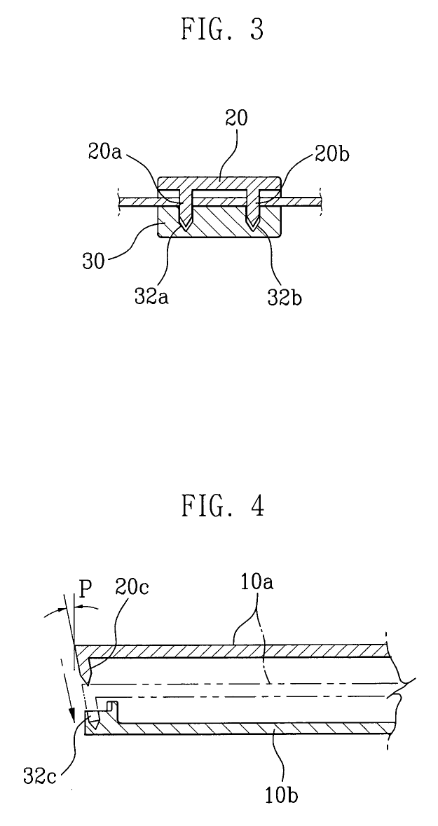

As illustrated in FIGS. 1 to 3, the present invention provides a forceps 100 resiliently regulated and including an upper support plate 20, a lower support plate 30, and handles 10a and 10b easily holdable with a hand.

The upper support plate 20 is attached to the front side of the handle 10a, and one or more needles 20a and 20b are formed to stand up in a front region of the inner surface of the upper support plate 20.

The needles 20a and 20b have an acute needle shape or a blade-like sharp and rectangular shape, but they are not intended to limit the scope of the present invention and will be able to be modified into any shapes for facilitating a double eyelid operation by a buried suture method.

A support member 60 corresponding to a curved upper eyelid margin is erected on a border between accommodation recesses 32a of the lower support plate 30 and the handle 10b such that the width between the needles 20a and 20b arranged on the upper support plate 20 and the support member 60 can be kept constant when the lower support plate 30 enters an eyelid and the needles 20a and 20b nip the eyelid.

The support member 60 is curved to correspond to the curved upper eyelid margin.

The lower support plate 30 corresponds to the upper support plate 20 and is attached to the front side of the handle 10b. The lower support plate 30 has one or more accommodation recesses 32a and 32b on the inner surface thereof.

The accommodation recesses 32a and 32b are formed to have the depth for accommodating the needles 20a and 20b in whole or in part or accommodating only a half of each needle 20a and 20b.

FIG. 4 is a longitudinal cross-sectional view illustrating main portions of a forceps according to another embodiment of the present invention. The needles 20a and 20b are erected, but needles 20c may be rather inclined.

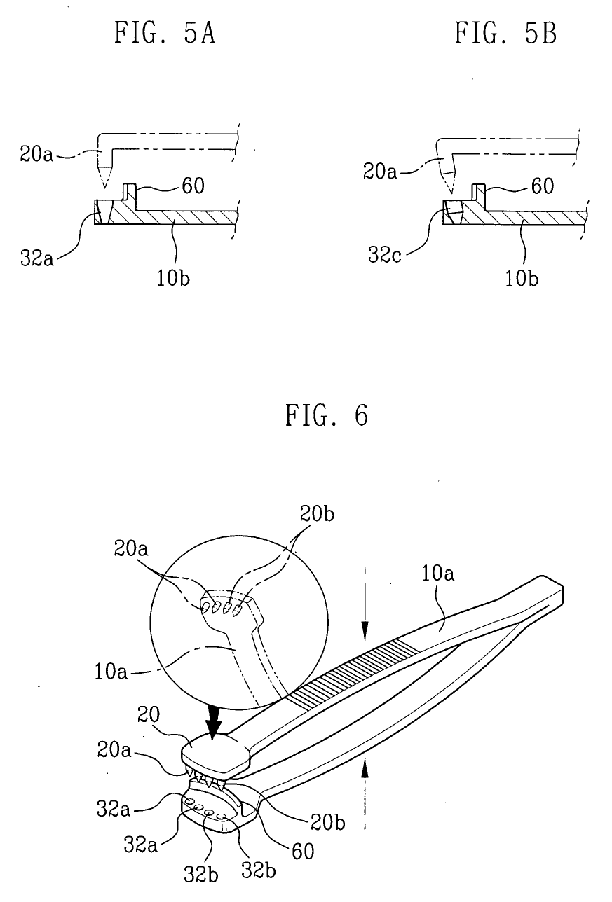

FIGS. 5A and 5B are longitudinal cross-sectional views of other embodiments of the present invention. The accommodation recesses 32a and 32c of the lower support plate 30 have vertical or oblique shapes and have blind-hole or through-hole interiors.

The upper support plate 20 and the lower support plate 30 have curved surfaces corresponding to the curved surface of an eyeball or have planar surfaces such that the lower support plate 30 can be inserted smoothly and concurrently, be matched with the upper support plate 30 without causing patients' discomfort during the operation.

The needles 20a and 20b provided in the upper support plate 20 have the width of about 0.5 mm to 3.0 mm and the length of about 1 mm to 10 mm. The needles 20a and 20b are of the length such that the needles 20a and 20b can be smoothly inserted into the accommodation recesses 32a and 32b of the lower support plate 20 when the forceps is folded. When the needles 20a and 20b are provided in plurality, they are arranged as many as required in the form of an oval or a line by the interval of about 2 mm to 6 mm. The lower support plate 30 has a thickness enough to smoothly insert the lower support plate 30 into the eyelid.

Operation of the double eyelid operation using the forceps according to the present invention is described below.

As shown in FIG. 1, the lower support plate 30 is inserted into the eyelid with the forceps 100 being unfolded. Then, the tips of the needles 20a and 20b of the upper support plate 20 are located at the desired portions of the eyelid that is to be punctured on the double eyelid line (designed line). Next, if the forceps is folded, the needles 20a and 20b penetrate the skin or the tarsal plate and concurrently, are accommodated in the accommodation recesses 32a and 32b formed in the lower support plate 30.

If the operation is completed, small holes are provided in the designed double eyelid line formed on the eyelid and punctured marks are easily made in the tarsal plate by the needles 20a and 20b.

The operation by the buried suture method is performed on the basis of the small holes of the double eyelid line and the punctured marks formed in the tarsal plate corresponding to the small holes. By doing so, the subcutis, the orbicularis oculi muscle, the levator aponeurosis, and the tarsal plate, which are positioned between the skin and the conjunctiva, are bound together with a thread to form a double eyelid. Here, since the buried suture method is a general double eyelid operation, the operation method is omitted.

According to the present invention, the operation time is so short and the operation method is so simple that even an unexperienced surgeon can safely and conveniently perform the operation and the time and costs can be saved to the modem Orientals to whom double eyelid operations are generalized.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

1. A forceps for a double eyelid operation that can be resiliently regulated by handles easily holdable by a hand, the forceps comprising:
an upper support plate being attached to the front side of the handle and having one or more needles arranged in a front region of the inner surface of the upper support plate; and
a lower support plate corresponding to the upper support plate, the lower support plate being attached to the front side of the handle and having accommodation recesses corresponding to the needles and being formed in a front region of the inner surface of the lower support plate.

2. The forceps according to claim 1, wherein a curved support member corresponding to a curved upper eyelid margin is erected on and attached to a border between the accommodation recesses of the lower support plate and the handle such that the width between the needles arranged on the upper support plate and the support member can be kept constant when the lower support plate enters an eyelid and the needles nip the eyelid.

3. The forceps according to claim 1, wherein the needles of the upper support plate are vertically formed from top to bottom or obliquely formed.

4. The forceps according to claim 1, wherein the accommodation recesses of the lower support plate are vertically formed or obliquely formed.

5. The forceps according to claim 1, wherein the accommodation recesses of the lower support plate have blind-hole shapes or through-hole shapes.

6. The forceps according to claim 1, wherein, when the needles of the upper support plate are plural, the needles are arranged linearly or curvedly.

7. The forceps according to claim 1, wherein the needles of the upper support plate are in the form of an awl or a cutting blade.

8. The forceps according to claim 1, wherein the needles of the upper support plate are in the form of an awl or a cutting blade alone or in the form of a combination thereof.
